(19) United States
(12) Patent Application Publication
(10) Pub. No.: US 2026/0134978 A1
(43) Pub. Date: May 14, 2026

(54) CONSTRUCTION METHOD OF ARTIFICIAL INTELLIGENCE (AI)-SIMULATED TEACHING MODEL FOR TEMPOROMANDIBULAR JOINT (TMJ) SURGERY

(71) Applicant: Sichuan University, Chengdu City (CN)

(72) Inventors: Ruiye BI, Chengdu City (CN); Haohan LI, Chengdu City (CN); Songsong ZHU, Chengdu City (CN); Yao LIU, Chengdu City (CN); Pinyin CAO, Chengdu City (CN); Xianni YANG, Chengdu City (CN); Liwei HUANG, Chengdu City (CN); Yiru WANG, Chengdu City (CN); Han FANG, Chengdu City (CN); Yanjing ZHAN, Chengdu City (CN); Ziqian WANG, Chengdu City (CN)

(21) Appl. No.: 19/388,500

(22) Filed: Nov. 13, 2025

(30) Foreign Application Priority Data
Nov. 13, 2024 (CN) 202411615042.6

(51) Int. Cl. G16H 30/20; G06T 3/40; G06T 7/00; G06T 7/11; G06T 7/13; G06V 10/75; G06V 10/764; G06V 20/70; G16H 30/40

(52) U.S. Cl. CPC G16H 30/20; G06T 3/40; G06T 7/0002; G06T 7/11; G06T 7/13; G06V 10/751; G06V 10/764; G06V 20/70; G16H 30/40; G06T 2207/10016; G06T 2207/30168

(57) ABSTRACT

Provided is a construction method of an artificial intelligence (AI)-simulated teaching model for temporomandibular joint (TMJ) surgery. The method includes: obtaining a plurality of key grayscale images of the TMJ surgical video according to differences between grayscale values of pixels in adjacent grayscale images of a TMJ surgical video; equally dividing each key grayscale image of the TMJ surgical video into a plurality of local regions; classifying the plurality of local regions into a plurality of labeled patches and a plurality of unlabeled patches; obtaining visual quality of each labeled patch in the key grayscale image of the TMJ surgical video according to differences between grayscale values of pixels in the labeled patches and the unlabeled patches in the key grayscale image of the TMJ surgical video; and performing resolution enhancement on the key grayscale image of the TMJ surgical video, and constructing an AI-simulated teaching model for TMJ surgery.

### CROSS REFERENCE TO RELATED APPLICATION

This patent application claims the benefit and priority of Chinese Patent Application No. 2024116150426, filed with the China National Intellectual Property Administration on Nov. 13, 2024, the disclosure of which is incorporated by reference herein in its entirety as part of the present application.

### TECHNICAL FIELD

The present disclosure relates to the field of image data processing, and in particular to a construction method of an artificial intelligence (AI)-simulated teaching model for temporomandibular joint (TMJ) surgery.

### BACKGROUND

The TMJ is one of the most complex joints in the human body, and it is essential for functions such as chewing, and speaking. The TMJ surgery encompasses various procedures like disc repair and joint replacement, and it is of great significance to treat joint diseases, reconstruct joint functions and improve patients' quality of life. At present, the TMJ surgery mainly relies on manual operation of experienced surgeons, and the surgical outcome largely depends on the doctor's experience and personal skills. Due to lack of effective simulation and training tools, young doctors tend to accumulate experience in actual surgeries, not only increasing surgical risks of the patients, and but also extending growth cycles of the doctors. With the rapid development of AI, by constructing AI-simulated teaching models for the TMJ surgery, learners can practice the surgical operation in a risk-free environment, and their performances can be evaluated and fed back in real time. During model construction, it is necessary to acquire clear images from a TMJ surgical video. Since the resolution of images captured from the video is reduced, the acquired images are not clear enough, and subtle structures and details cannot be captured accurately in the model construction. Consequently, the teaching models have a certain error, and cannot effectively improve the safety and success rate of the surgery.

### SUMMARY

The present disclosure provides a construction method of an AI-simulated teaching model for TMJ surgery, to solve the problem that since the resolution of images captured from a video is reduced, the acquired images are not clear enough, and subtle structures and details cannot be captured accurately in the model construction, causing a certain error to the constructed teaching model.

The construction method of an AI-simulated teaching model for TMJ surgery in the present disclosure adopts the following technical solutions.

An embodiment of the present disclosure provides a construction method of an AI-simulated teaching model for TMJ surgery, including the following steps:
acquiring a plurality of grayscale images of a TMJ surgical video and a corresponding time node for each grayscale image of the TMJ surgical video;
obtaining a plurality of key grayscale images of the TMJ surgical video according to differences between grayscale values of pixels in adjacent grayscale images of the TMJ surgical video;
equally dividing each key grayscale image of the TMJ surgical video into a plurality of local regions, and classifying the plurality of local regions into a plurality of labeled patches and a plurality of unlabeled patches;
obtaining visual quality of each labeled patch in the key grayscale image of the TMJ surgical video according to differences between grayscale values of pixels in the labeled patches and the unlabeled patches in the key grayscale image of the TMJ surgical video;
segmenting a TMJ cartilaginous region in the key grayscale image of the TMJ surgical video, and obtaining a contrast enhancement requirement for the local regions in the key grayscale image of the TMJ surgical video according to slopes of straight lines passing through two adjacent pixels on a contour boundary line of a surface of the TMJ cartilaginous region and the visual quality of the labeled patch;
obtaining a resolution enhancement credibility for each key grayscale image of the TMJ surgical video according to the contrast enhancement requirement for the local regions in the key grayscale image of the TMJ surgical video and the corresponding time node for each grayscale image of the TMJ surgical video; and
performing resolution enhancement on the key grayscale image of the TMJ surgical video according to the resolution enhancement credibility for each key grayscale image of the TMJ surgical video, and constructing an AI-simulated teaching model for TMJ surgery.

Preferably, the obtaining a plurality of key grayscale images of the TMJ surgical video according to differences between grayscale values of pixels in adjacent grayscale images of the TMJ surgical video specifically includes:
obtaining an information difference between the adjacent grayscale images of the TMJ surgical video according to the differences between the grayscale values of the pixels in the adjacent grayscale images of the TMJ surgical video and a difference between information entropies for the grayscale values of the pixels; and
obtaining the plurality of key grayscale images of the TMJ surgical video according to the information difference between the adjacent grayscale images of the TMJ surgical video.

Preferably, the obtaining an information difference between the adjacent grayscale images of the TMJ surgical video according to the differences between the grayscale values of the pixels in the adjacent grayscale images of the TMJ surgical video and a difference between information entropies for the grayscale values of the pixels is specifically implemented based on the following equation:

$${JV}_{i , i + 1} = \frac{1}{n} {\sum }_{j = 1}^{n} \vert {I}_{j}^{i} - {I}_{j}^{i + 1} \vert \times \vert {H}_{i} - {H}_{i + 1} \vert$$

where, JV_{i, i+1} represents an information difference between an i^{th} grayscale image and an (i+1)^{th} grayscale image of the TMJ surgical video; n represents a quantity of all pixels in the grayscale images of the TMJ surgical video;

$${I}_{j}^{i}$$

represents a grayscale value of a j^{th} pixel in the i^{th} grayscale image of the TMJ surgical video;

$${I}_{j}^{i + 1}$$

represents a grayscale value of a j^{th} pixel in the (i+1)^{th} grayscale image of the TMJ surgical video; Hᵢ represents an information entropy for gray values of all pixels in the i^{th} grayscale image of the TMJ surgical video; Hᵢ₊₁ represents an information entropy for gray values of all pixels in the (i+1)^{th} grayscale image of the TMJ surgical video; and ∥ is an absolute value function.

Preferably, the obtaining the plurality of key grayscale images of the TMJ surgical video according to the information difference between the adjacent grayscale images of the TMJ surgical video specifically includes:
in all grayscale images of the TMJ surgical video, sorting all information differences between two adjacent grayscale images of the TMJ surgical video in an ascending manner to obtain an information difference sequence; and
in the information difference sequence, calculating an absolute value of a difference between two adjacent information differences, and labeling two grayscale images of the TMJ surgical video corresponding to a former information difference in two information differences that correspond to a maximum value in all absolute values of differences between adjacent information differences as key grayscale images of the TMJ surgical video.

Preferably, the classifying the plurality of local regions into a plurality of labeled patches and a plurality of unlabeled patches specifically includes:
acquiring a plurality of shape template images of a surgical instrument;
respectively matching all shape template images of the surgical instrument with the plurality of local regions of the key grayscale image of the TMJ surgical video by template matching to obtain local regions with the surgical instrument in each key grayscale image of the TMJ surgical video, and labeling the local regions with the surgical instrument as target local regions of the surgical instrument; and
from the target local regions of the surgical instrument, selecting target local regions of the surgical instrument containing the TMJ cartilaginous region, and labeling the target local regions of the surgical instrument containing the TMJ cartilaginous region as the labeled patches;
and labeling target local regions of the surgical instrument not containing the TMJ cartilaginous region as the unlabeled patches.

Preferably, the obtaining visual quality of each labeled patch in the key grayscale image of the TMJ surgical video according to differences between grayscale values of pixels in the labeled patches and the unlabeled patches in the key grayscale image of the TMJ surgical video is specifically implemented based on the following equation:

$${JH}_{e}^{v} = {\sum }_{f = 1}^{{U}^{v}} \text{\textbar} {\overline{{h}_{e}}}^{v} - {\overline{{h}_{f}}}^{v} \vert$$

where, JHₑ^{v} represents visual quality of an e^{th} labeled patch in a v^{th} key grayscale image of the TMJ surgical video; U^{v} represents a quantity of all unlabeled patches in the v^{th} key grayscale image of the TMJ surgical video; h̅ₑ̅^{v} represents an average grayscale value for all pixels in the e^{th} labeled patch in the v^{th} key grayscale image of the TMJ surgical video; h̅_{f}^{v} represents an average grayscale value for all pixels in an f^{th} unlabeled patch in the v^{th} key grayscale image of the TMJ surgical video; and ∥ is an absolute value function.

Preferably, the obtaining a contrast enhancement requirement for the local regions in the key grayscale image of the TMJ surgical video according to slopes of straight lines passing through two adjacent pixels on a contour boundary line of a surface of the TMJ cartilaginous region and the visual quality of the labeled patch specifically includes:
performing edge detection on the key grayscale image of the TMJ surgical video to obtain a plurality of edge pixels;
obtaining a slope sequence and a quantity of breakpoints according to the slopes of the straight lines passing through the two adjacent pixels on the contour boundary line of the surface of the TMJ cartilaginous region and the edge pixels; and
obtaining the contrast enhancement requirement for the local regions in the key grayscale image of the TMJ surgical video according to the quantity of breakpoints on the contour boundary line of the surface of the TMJ cartilaginous region, the slope sequence, and the visual quality of the labeled patch.

Preferably, the obtaining a slope sequence and a quantity of breakpoints according to the slopes of the straight lines passing through the two adjacent pixels on the contour boundary line of the surface of the TMJ cartilaginous region and the edge pixels specifically includes:
labeling a quantity of pixels, not being the edge pixels, on the contour boundary line of the surface of the TMJ cartilaginous region as the quantity of breakpoints; and
in the key grayscale image of the TMJ surgical video, from any pixel on the contour boundary line of the surface of the TMJ cartilaginous region, along a clockwise direction, counting slopes of straight lines passing through two adjacent pixels in sequence to obtain the slope sequence.

Preferably, the obtaining the contrast enhancement requirement for the local regions in the key grayscale image of the TMJ surgical video according to the quantity of breakpoints on the contour boundary line of the surface of the TMJ cartilaginous region, the slope sequence, and the visual quality of the labeled patch is specifically implemented based on the following equation:

$${JB}_{v} = \frac{1}{N - 1} {\sum }_{r = 1}^{N - 1} \vert {K}_{v , r} - {K}_{v , r + 1} \vert \times \frac{{G}^{v} \times {g}^{v}}{{\sum }_{e = 1}^{g} {JH}_{e}^{v}}$$

where, JBᵥ represents a contrast enhancement requirement for local regions in a v^{th} key grayscale image of the TMJ surgical video; JHₑ^{v} represents visual quality of an e^{th} labeled patch in the v^{th} key grayscale image of the TMJ surgical video; g^{v} represents a quantity of all labeled patches in the v^{th} key grayscale image of the TMJ surgical video; G^{v} represents a quantity of breakpoints on a contour boundary line of a TMJ cartilaginous region in the v^{th} key grayscale image of the TMJ surgical video; K_{v,r} represents an r^{th} slope in a slope sequence corresponding to the v^{th} key grayscale image of the TMJ surgical video; K_{v,r+1} represents an (r+1)^{th} slope in the slope sequence corresponding to the v^{th} key grayscale image of the TMJ surgical video; N represents a quantity of all slopes in the slope sequence corresponding to the v^{th} key grayscale image of the TMJ surgical video; and ∥ is an absolute value function.

Preferably, the obtaining a resolution enhancement credibility for each key grayscale image of the TMJ surgical video according to the contrast enhancement requirement for the local regions in the key grayscale image of the TMJ surgical video and the corresponding time node for each grayscale image of the TMJ surgical video is specifically implemented based on the following equation:

$${JL}_{v} = {JB}_{v} \times \{ 1 + th [ ( {t}_{v} - {t}_{s} ) \times ( {t}_{a} - {t}_{v} ) ] \}$$

where, JLᵥ represents a resolution enhancement credibility of a v^{th} key grayscale image of the TMJ surgical video; JBᵥ represents a contrast enhancement requirement for local regions in the v^{th} key grayscale image of the TMJ surgical video; tᵥ represents a time node for the v^{th} key grayscale image of the TMJ surgical video; tₛ represents a time node for a first grayscale image of the TMJ surgical video; tₐ represents a time node for a last grayscale image of the TMJ surgical video; and th[ ] is a hyperbolic tangent function.

The technical solutions of the present disclosure have the following beneficial effects: According to differences between grayscale values of pixels in adjacent grayscale images of a TMJ surgical video, a plurality of key grayscale images of the TMJ surgical video are obtained. Each key grayscale image of the TMJ surgical video is equally divided into a plurality of local regions. All local regions are classified into a plurality of labeled patches and a plurality of unlabeled patches. According to differences between grayscale values of pixels in the labeled patches and the unlabeled patches in the key grayscale image of the TMJ surgical video, visual quality of each labeled patch in the key grayscale image of the TMJ surgical video is obtained. The present disclosure provides a data support for construction of the AI teaching model for the TMJ surgery, ensuring that the teaching model can restore details of the real surgical process to the maximum extent in use. According to a contrast enhancement requirement for the local regions in the key grayscale image of the TMJ surgical video and a corresponding time node for each grayscale image of the TMJ surgical video, a resolution enhancement credibility for each key grayscale image of the TMJ surgical video is obtained. According to the resolution enhancement credibility for each key grayscale image of the TMJ surgical video, resolution enhancement is performed on the key grayscale image of the TMJ surgical video, and an AI-simulated teaching model is constructed for the TMJ surgery. The present disclosure improves the accuracy of the AI-simulated teaching model for the TMJ surgery, providing an efficient, safe, and reliable surgical learning platform for medical students and doctors.

### BRIEF DESCRIPTION OF THE DRAWINGS

To describe the technical solutions in the embodiments of the present disclosure or in the prior art more clearly, the following briefly describes the accompanying drawings required for describing the embodiments or the prior art. Apparently, the accompanying drawings in the following description show merely some embodiments of the present disclosure, and a person of ordinary skill in the art may still derive other drawings from these accompanying drawings without creative efforts.

FIG. 1 is a flowchart of a construction method of an AI-simulated teaching model for TMJ surgery according to the present disclosure.

### DETAILED DESCRIPTION OF THE EMBODIMENTS

To further describe the adopted technical means and the effects of the present disclosure to achieve an intended purpose of the present disclosure, the following describes specific implementations, structures, features, and effects of a construction method of an AI-simulated teaching model for TMJ surgery according to the present disclosure in detail with reference to the accompanying drawings and preferred embodiments. In the following description, different references to “an embodiment” or “another embodiment” are not necessarily to the same embodiment. In addition, particular features, structures, or characteristics in one or more embodiments may be combined in any suitable form.

Unless otherwise defined, all technical and scientific terms used herein have the same meanings as commonly understood by those skilled in the technical field of the present disclosure.

Specific solutions of the construction method of an AI-simulated teaching model for TMJ surgery according to the present disclosure are described below with reference to the accompanying drawings.

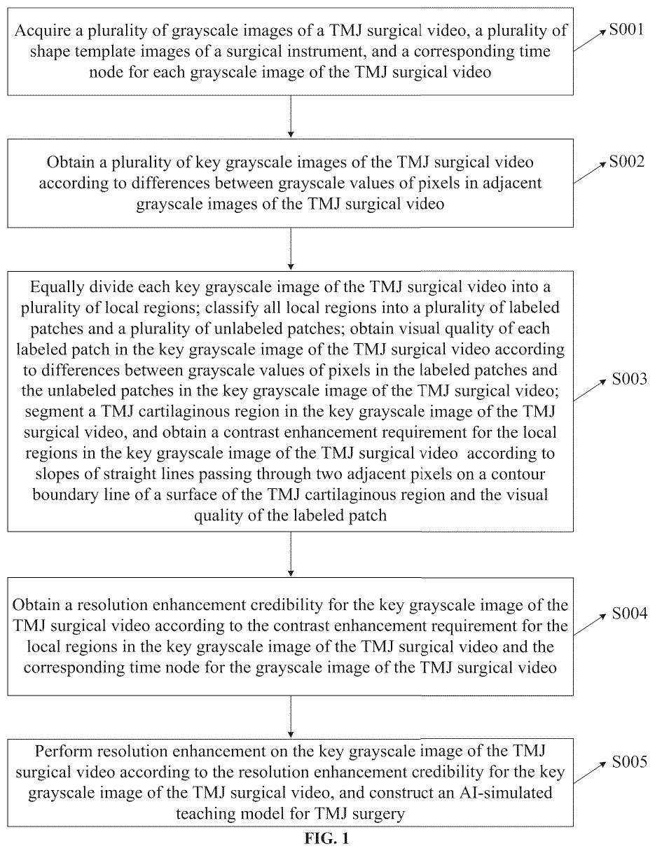

FIG. 1 is a flowchart of a construction method of an AI-simulated teaching model for TMJ surgery according to an embodiment of the present disclosure. The method includes the following steps.

Step S001: A plurality of grayscale images of a TMJ surgical video, a plurality of shape template images of a surgical instrument, and a corresponding time node for each grayscale image of the TMJ surgical video are acquired.

It is to be noted that construction of a teaching model is a complex and systematic process. First of all, an objective and a scope of the teaching model, including a target population, a teaching content, and a teaching outcome evaluation standard, are determined. Then, related data of TMJ surgery, including a surgical video, anatomical model data, medical literature and the like, is acquired. Next, a structure and a function of the teaching model are designed, and according to a designed structure of the teaching model, an appropriate technique and an appropriate tool are used to develop the teaching model. At last, a developed teaching model is integrated to a teaching platform or device, and deployed.

In a disclosed TMJ surgical video, grayscale processing is performed on each image of the TMJ surgical video, to obtain a plurality of grayscale images of the TMJ surgical video and a corresponding time node for each grayscale image of the TMJ surgical video.

A plurality of shape template images of a surgical instrument are acquired in a medical database.

It is to be noted that when the images of the surgical video are acquired, attention should be paid to protect privacy and data security of a patient, ensuring that the process of acquiring the images of the video is legal, without violating the privacy right of the patient.

Step S002: A plurality of key grayscale images of the TMJ surgical video are obtained according to differences between grayscale values of pixels in adjacent grayscale images of the TMJ surgical video.

It is to be noted that in the construction method of an AI-simulated teaching model for TMJ surgery, by enhancing the contrast and luminance of the image of the video, key details in the surgical process, such as a surgical tool, a tissue structure, and a boundary of a surgical region, can be distinguished by the model more easily. With the image enhancement technique, diverse training data can be generated, thereby improving the generalization capability of the model, effectively improving the visual experience of learners, and enabling the learners to better understand the complexity and details in the surgical process.

It is to be noted that the TMJ surgery encompasses various procedures, and each procedure corresponds to one lesion of the TMJ. According to various surgical videos in the resource library, the surgical video of one procedure is selected as an analysis object. The surgical video is divided into a plurality of images based on frames.

An information entropy for grayscale values of pixels is obtained based on an equation for calculating the entropy. The equation for calculating the entropy is the well-known technique, and the specific method is not described herein.

It is to be noted that key images are extracted from the grayscale images of the TMJ surgical video, and these key images represent main operations and changes in a short time period.

An information difference between an i^{th} grayscale image and an (i+1)^{th} grayscale image of the TMJ surgical video is calculated by:

$${JV}_{i , i + 1} = \frac{1}{n} {\sum }_{j = 1}^{n} \vert {I}_{j}^{i} - {I}_{j}^{i + 1} \vert \times \vert {H}_{i} - {H}_{i + 1} \vert$$

where, JV_{i, i+1} represents the information difference between the i^{th} grayscale image and the (i+1)^{th} grayscale image of the TMJ surgical video; n represents a quantity of all pixels in the grayscale images of the TMJ surgical video;

$${I}_{j}^{i}$$

represents a grayscale value of a j^{th} pixel in the i^{th} grayscale image of the TMJ surgical video;

$${I}_{j}^{i + 1}$$

represents a grayscale value of a j^{th} pixel in the (i+1)^{th} grayscale image of the TMJ surgical video; Hᵢ represents an information entropy for gray values of all pixels in the i^{th} grayscale image of the TMJ surgical video; Hᵢ₊₁ represents an information entropy for gray values of all pixels in the (i+1)^{th} grayscale image of the TMJ surgical video; and ∥ is an absolute value function.

It is to be noted that

$$\frac{1}{n} {\sum }_{j = 1}^{n} \vert {I}_{j}^{i} - {I}_{j}^{i + 1} \vert$$

represents an average of grayscale differences between all pixels at same positions in two adjacent grayscale images of the TMJ surgical video, and a greater average indicates a significant difference between the pixels at the same positions in the two grayscale images, namely the two adjacent grayscale images are varied greatly; ∥Hᵢ−Hᵢ₊₁| represents a difference between information entropies for grayscale values of the pixels in the two adjacent grayscale images, and a greater difference indicates a significant distribution difference between the pixels in the two adjacent grayscale images; and

$$\frac{1}{n} {\sum }_{j = 1}^{n} \vert {I}_{j}^{i} - {I}_{j}^{i + 1} \vert \times \vert {H}_{i} - {H}_{i + 1} \vert$$

represents an information difference between the two adjacent grayscale images, and a greater product indicates more information differences between the two adjacent grayscale images, namely significant changes in the surgical video, such as in key steps or anatomical structures in surgical operation, can increase the information difference between the adjacent grayscale images.

Therefore, all information differences between two adjacent grayscale images of the TMJ surgical video are obtained.

In all grayscale images of the TMJ surgical video, the all information differences between the two adjacent grayscale images of the TMJ surgical video are sorted in an ascending manner to obtain an information difference sequence.

In the information difference sequence, an absolute value of a difference between two adjacent information differences is calculated, and two grayscale images of the TMJ surgical video corresponding to a former information difference in two information differences that correspond to a maximum value in all absolute values of differences between adjacent information differences is labeled as key grayscale images of the TMJ surgical video.

It is to be noted that if there are a plurality of maximum values, a first maximum value in the information difference sequence is used.

Therefore, a plurality of key grayscale images of the TMJ surgical video are obtained.

Step S003: Each key grayscale image of the TMJ surgical video is equally divided into a plurality of local regions; all local regions are classified into a plurality of labeled patches and a plurality of unlabeled patches; according to differences between grayscale values of pixels in the labeled patches and the unlabeled patches in the key grayscale image of the TMJ surgical video, visual quality of each labeled patch in the key grayscale image of the TMJ surgical video is obtained; a TMJ cartilaginous region in the key grayscale image of the TMJ surgical video is segmented, and according to slopes of straight lines passing through two adjacent pixels on a contour boundary line of the surface of the TMJ cartilaginous region and the visual quality of the labeled patch, a contrast enhancement requirement for the local regions in the key grayscale image of the TMJ surgical video is obtained.

It is to be noted that by enhancing a contrast of the key grayscale image of the TMJ surgical video, surgical details and structures can be displayed more clearly. Highlighting the contrast of local details and regions is particularly beneficial for enhancing details such as instruments, tissue boundaries, and blood vessels in the surgical video.

Edge detection is performed on the key grayscale image of the TMJ surgical video to obtain a plurality of edge pixels. The Canny edge detection algorithm is used to perform the edge detection, which is the well-known technique.

In the embodiment of the present disclosure, the TMJ cartilaginous region in the key grayscale image of the TMJ surgical video is recognized using a segmentation neural network.

The related content of the segmentation neural network is as follows.

The segmentation neural network used in the embodiment is a Mask region-based convolutional neural network (R-CNN). The datasets used are datasets for all key grayscale images of the TMJ surgical video. The Mask R-CNN is the well-known technique, and the specific method is not described herein. The Mask R-CNN stands for the mask region-based convolutional neural network.

Pixels to be segmented are classified into two classes. That is, corresponding labels in a training set are annotated as follows: Single-channel semantic labels are used, pixels in the normal region are annotated as 0, and pixels in the TMJ cartilaginous region are annotated as 1.

To classify the network, a cross-entropy loss function is used as the loss function.

The process for obtaining the TMJ cartilaginous region in the key grayscale image of the TMJ surgical video through the segmentation neural network is the well-known technique, and the specific method is not described herein.

A quantity of pixels, not being the edge pixels, on the contour boundary line of the surface of the TMJ cartilaginous region is labeled as a quantity of breakpoints.

In the key grayscale image of the TMJ surgical video, from any pixel on the contour boundary line of the surface of the TMJ cartilaginous region, along a clockwise direction, slopes of straight lines passing through two adjacent pixels are counted in sequence to obtain a slope sequence.

A v^{th} key grayscale image of the TMJ surgical video is equally divided into a plurality of local regions. A case where the local regions are square and there are 20 local regions is taken as an example for description.

All shape template images of the surgical instrument are respectively matched with all local regions of the key grayscale image of the TMJ surgical video by template matching to obtain local regions with the surgical instrument in each key grayscale image of the TMJ surgical video, and the local regions with the surgical instrument are labeled as target local regions of the surgical instrument.

From the target local regions of the surgical instrument, target local regions of the surgical instrument containing the TMJ cartilaginous region are selected, and the target local regions of the surgical instrument with the TMJ cartilaginous region are labeled as the labeled patches; and target local regions of the surgical instrument not containing the TMJ cartilaginous region are labeled as the unlabeled patches.

The template matching technique is the well-known technique, and the specific method is not described herein.

Visual quality of an e^{th} labeled patch in the v^{th} key grayscale image of the TMJ surgical video is calculated by:

$$J {H}_{e}^{v} = {\sum }_{f = 1}^{{U}^{v}} \vert {\overline{{h}_{e}}}^{v} - {\overline{{h}_{f}}}^{v} \vert$$

where, JHₑ^{v} represents the visual quality of the v^{th} labeled patch in the e^{th} key grayscale image of the TMJ surgical video; U^{v} represents a quantity of all unlabeled patches in the v^{th} key grayscale image of the TMJ surgical video; h̅ₑ̅^{v} represents an average grayscale value for all pixels in the e^{th} labeled patches in the v^{th} key grayscale image of the TMJ surgical video; h̅_{f}^{v} represents an average grayscale value for all pixels in an f^{th} unlabeled patches in the v^{th} key grayscale image of the TMJ surgical video; and ∥ is an absolute value function.

It is to be noted that the U^{v} represents the quantity of all unlabeled patches in the v^{th} key grayscale image of the TMJ surgical video,

$${\sum }_{f = 1}^{{U}^{v}} \vert {\overline{{h}_{e}}}^{v} - {\overline{{h}_{f}}}^{v} \vert$$

represents a sum of grayscale differences between pixels in the e^{th} labeled patch and an f^{th} unlabeled patch in the v^{th} key grayscale image of the TMJ surgical video, and a higher value indicates a significant grayscale difference between the labeled patch and the unlabeled patch in this image, and the labeled patch has a prominent luminance in this image, namely higher visual quality.

A contrast enhancement requirement for the local regions in the v^{th} key grayscale image of the TMJ surgical video is calculated by:

$$J {B}_{v} = \frac{1}{N - 1} {\sum }_{r = 1}^{N - 1} \vert {K}_{v , r} - {K}_{v , r + 1} \vert \times \frac{{G}^{v} \times {g}^{v}}{{\sum }_{e = 1}^{g} J {H}_{e}^{v}}$$

where, JBᵥ represents the contrast enhancement requirement for the local regions in the v^{th} key grayscale image of the TMJ surgical video; JHₑ^{v} represents visual quality of an e^{th} labeled patch in the v^{th} key grayscale image of the TMJ surgical video; g^{v} represents a quantity of all labeled patches in the v^{th} key grayscale image of the TMJ surgical video; G^{v} represents a quantity of breakpoints on a contour boundary line of a TMJ cartilaginous region in the v^{th} key grayscale image of the TMJ surgical video; K_{v,r} represents an r^{th} slope in a slope sequence corresponding to the v^{th} key grayscale image of the TMJ surgical video; K_{v,r+1} represents an (r+1)^{th} slope in the slope sequence corresponding to the v^{th} key grayscale image of the TMJ surgical video; N represents a quantity of all slopes in the slope sequence corresponding to the v^{th} key grayscale image of the TMJ surgical video; and ∥ is an absolute value function.

It is to be noted that when the denominator in the equation is 0, to ensure that the equation is valid, a case where the denominator is set as 1 is used as an example for description.

It is to be noted that

$$\frac{1}{N - 1} {\sum }_{r = 1}^{N - 1} \vert {K}_{v , r} - {K}_{v , r + 1} \vert$$

represent a flatness of the contour edge line of the TMJ cartilaginous region in the key grayscale image of the TMJ surgical video, and a greater average indicates a more uneven contour edge line. In a low-contrast image, the contour edge line of the cartilage is not clear enough in the image, thus causing the unevenness. Gᵥ represents a quantity of breakpoints on the edge line, and the more breakpoints indicate that the edge line is more discontinuous. However, the discontinuous contour edge line of the TMJ cartilaginous region in the key grayscale image typically implies a low contrast of the image.

$$\frac{{\sum }_{e = 1}^{g} {JH}_{e}^{v}}{{g}^{v}}$$

represents an average visual quality indicator for all labeled patches in the grayscale image, and a smaller average indicates a smaller information difference between the labeled patch and the unlabeled patch, namely lower visual quality.

$$\frac{1}{N - 1} {\sum }_{r = 1}^{N - 1} \vert {K}_{v , r} - {K}_{v , r + 1} \vert \times \frac{{G}^{v} \times {g}^{v}}{{\sum }_{e = 1}^{g} {JH}_{e}^{v}}$$

represents the contrast enhancement requirement for the local regions in the key grayscale image, and a greater product indicates a low contrast for the local regions in the key grayscale image, such that a higher contrast is required to highlight the key image.

Therefore, the contrast enhancement requirement for the local regions in the key grayscale image of the TMJ surgical video is obtained.

Step S004: A resolution enhancement credibility for each key grayscale image of the TMJ surgical video is obtained according to the contrast enhancement requirement for the local regions in the key grayscale image of the TMJ surgical video and the corresponding time node for each grayscale image of the TMJ surgical video.

First of all, time nodes for all grayscale images of the TMJ surgical video are acquired. A time node for a first grayscale image is labeled as tₛ, and a time node for a last grayscale image is labeled as tₐ.

A time node for each key grayscale image of the TMJ surgical video is recorded.

A resolution enhancement credibility for the v^{th} key grayscale image of the TMJ surgical video is calculated by:

$$J {L}_{v} = J {B}_{v} \times \{ 1 + t h [ ( {t}_{v} - {t}_{s} ) \times ( {t}_{a} - {t}_{v} ) ] \}$$

where, JLᵥ represents the resolution enhancement credibility of the v^{th} key grayscale image of the TMJ surgical video; JBᵥ represents a contrast enhancement requirement for local regions in the v^{th} key grayscale image of the TMJ surgical video; tᵥ represents a time node for the v^{th} key grayscale image of the TMJ surgical video; tₛ represents the time node for the first grayscale image of the TMJ surgical video; tₐ represents the time node for the last grayscale image of the TMJ surgical video; and th[ ] is a hyperbolic tangent function.

It is to be noted that (tᵥ−tₛ) represents a difference between the time node for the v^{th} key grayscale image of the TMJ surgical video and the time node for the first grayscale image of the TMJ surgical video. (tₐ−tᵥ) represents a difference between the time node for the last grayscale image of the TMJ surgical video and the time node for the v^{th} key grayscale image of the TMJ surgical video. th[(tᵥ−tₛ)×(tₐ−tᵥ)] represents an adaptive interpolation weight for the key grayscale image of the TMJ surgical video, and a greater product indicates that the time node for the key grayscale image is further from a time node for the start of the surgery and a time node for the end of the surgery, and the time node for the key grayscale image is in the surgery. For the surgery at this time node, changes of details are particularly important, and a higher interpolation weight is required. JBᵥ×{1+th[(tᵥ−tₛ)×(tₐ−tᵥ)]}represents the resolution enhancement credibility for the key grayscale image, and a greater product indicates a higher resolution enhancement credibility for the key grayscale image, namely the resolution of the key grayscale image can better highlight the changes of the details in the surgery.

Therefore, the resolution enhancement credibility for each key grayscale image of the TMJ surgical video is obtained.

Step S005: Resolution enhancement is performed on the key grayscale image of the TMJ surgical video according to the resolution enhancement credibility for each key grayscale image of the TMJ surgical video, and an AI-simulated teaching model for TMJ surgery is constructed.

According to the resolution enhancement credibility for each key grayscale image of the TMJ surgical video, the resolution enhancement is performed on the key grayscale image of the TMJ surgical video with an image pyramid, and the AI-simulated teaching model for the TMJ surgery is constructed with finite element simulation.

The image pyramid and the finite element simulation are the well-known techniques, and their specific methods are not described herein.

It is to be noted that for all key grayscale images of the TMJ surgical video, blank spaces are respectively added to adjacent rows and adjacent columns in the image matrix, and to ensure that the images have a same size, a Gaussian kernel is taken as an interpolation kernel in the adaptive interpolation. The resolution enhancement credibility for each key grayscale image of the TMJ surgical video is taken as a weight of the interpolation kernel to complete adaptive interpolation, thereby enhancing a resolution of the key image of the TMJ surgical video, and obtaining a high-resolution grayscale image of the TMJ surgical video. In combination with surgical actions and steps of a doctor, the model is established with simulation, and integrated to interactive learning by augmented reality (AR).

It is to be noted that with the Gaussian kernel, a group of weights following a standard deviation are generated for a convolution kernel, and then taken as weights to perform convolution on blank spaces inserted into the key grayscale image of the TMJ surgical video and neighboring pixels, thereby obtaining values of pixels represented by the inserted blank spaces. The adaptive interpolation on the key grayscale image can enhance the resolution of the key image. The convolution kernel of a Gaussian filter in the Gaussian filtering is the Gaussian kernel. The Gaussian kernel is the well-known technique, and the specific method is not described herein.

Therefore, this embodiment is completed.

The above described are merely preferred embodiments of the present disclosure, and not intended to limit the present disclosure. Any modifications, equivalent replacements and improvements made within the spirit and principle of the present disclosure should all fall within the scope of protection of the present disclosure.

What is claimed is:

1. A construction method of an artificial intelligence (AI)-simulated teaching model for temporomandibular joint (TMJ) surgery, comprising the following steps:
acquiring a plurality of grayscale images of a TMJ surgical video and a corresponding time node for each grayscale image of the TMJ surgical video;
obtaining a plurality of key grayscale images of the TMJ surgical video according to differences between grayscale values of pixels in adjacent grayscale images of the TMJ surgical video;
equally dividing each key grayscale image of the TMJ surgical video into a plurality of local regions, and classifying the plurality of local regions into a plurality of labeled patches and a plurality of unlabeled patches;
obtaining visual quality of each labeled patch in the key grayscale image of the TMJ surgical video according to differences between grayscale values of pixels in the labeled patches and the unlabeled patches in the key grayscale image of the TMJ surgical video;
segmenting a TMJ cartilaginous region in the key grayscale image of the TMJ surgical video, and according to slopes of straight lines passing through two adjacent pixels on a contour boundary line of a surface of the TMJ cartilaginous region and the visual quality of the labeled patch, obtaining a contrast enhancement requirement for the plurality of local regions in the key grayscale image of the TMJ surgical video;
obtaining a resolution enhancement credibility for each key grayscale image of the TMJ surgical video according to the contrast enhancement requirement for the plurality of local regions in the key grayscale image of the TMJ surgical video and the corresponding time node for each grayscale image of the TMJ surgical video; and
performing resolution enhancement on the key grayscale image of the TMJ surgical video according to the resolution enhancement credibility for each key grayscale image of the TMJ surgical video, and constructing an AI-simulated teaching model for TMJ surgery, wherein
the classifying the plurality of local regions into a plurality of labeled patches and a plurality of unlabeled patches specifically comprises:
acquiring a plurality of shape template images of a surgical instrument;
respectively matching all shape template images of the surgical instrument with the plurality of local regions of the key grayscale image of the TMJ surgical video by template matching to obtain local regions with the surgical instrument in each key grayscale image of the TMJ surgical video, and labeling the local regions with the surgical instrument as target local regions of the surgical instrument; and
from the target local regions of the surgical instrument, selecting target local regions of the surgical instrument containing the TMJ cartilaginous region, and labeling the target local regions of the surgical instrument containing the TMJ cartilaginous region as the labeled patches; and labeling target local regions of the surgical instrument not containing the TMJ cartilaginous region as the unlabeled patches;
the obtaining a contrast enhancement requirement for the plurality of local regions in the key grayscale image of the TMJ surgical video according to slopes of straight lines passing through two adjacent pixels on a contour boundary line of a surface of the TMJ cartilaginous region and the visual quality of the labeled patch specifically comprises:
performing edge detection on the key grayscale image of the TMJ surgical video to obtain a plurality of edge pixels;
obtaining a slope sequence and a quantity of breakpoints according to the slopes of the straight lines passing through the two adjacent pixels on the contour boundary line of the surface of the TMJ cartilaginous region and the edge pixels; and
obtaining the contrast enhancement requirement for the plurality of local regions in the key grayscale image of the TMJ surgical video according to the quantity of breakpoints on the contour boundary line of the surface of the TMJ cartilaginous region, the slope sequence, and the visual quality of the labeled patch;
the obtaining a slope sequence and a quantity of breakpoints according to the slopes of the straight lines passing through the two adjacent pixels on the contour boundary line of the surface of the TMJ cartilaginous region and the edge pixels specifically comprises:
labeling a quantity of pixels, not being the edge pixels, on the contour boundary line of the surface of the TMJ cartilaginous region as the quantity of breakpoints; and
in the key grayscale image of the TMJ surgical video, from any pixel on the contour boundary line of the surface of the TMJ cartilaginous region, along a clockwise direction, counting slopes of straight lines passing through two adjacent pixels in sequence to obtain the slope sequence;
the obtaining the contrast enhancement requirement for the plurality of local regions in the key grayscale image of the TMJ surgical video according to the quantity of breakpoints on the contour boundary line of the surface of the TMJ cartilaginous region, the slope sequence, and the visual quality of the labeled patch is specifically implemented based on the following equation:
$$J {B}_{v} = \frac{1}{N - 1} {\sum }_{r = 1}^{N - 1} \vert {K}_{v , r} - {K}_{v , r + 1} \vert \times \frac{{G}^{v} \times {g}^{v}}{{\sum }_{e = 1}^{g} J {H}_{e}^{v}}$$
wherein, JBᵥ represents a contrast enhancement requirement for local regions in a v^{th} key grayscale image of the TMJ surgical video; JHₑ^{v} represents visual quality of an e^{th} labeled patch in the v^{th} key grayscale image of the TMJ surgical video; g^{v} represents a quantity of all labeled patches in the v^{th} key grayscale image of the TMJ surgical video; G^{v} represents a quantity of breakpoints on a contour boundary line of a TMJ cartilaginous region in the v^{th} key grayscale image of the TMJ surgical video; K_{v,r} represents an r^{th} slope in a slope sequence corresponding to the v^{th} key grayscale image of the TMJ surgical video; K_{v,r+1} represents an (r+1)^{th} slope in the slope sequence corresponding to the v^{th} key grayscale image of the TMJ surgical video; N represents a quantity of all slopes in the slope sequence corresponding to the v^{th} key grayscale image of the TMJ surgical video; and ∥ is an absolute value function; and
the obtaining a resolution enhancement credibility for the key grayscale image of the TMJ surgical video according to the contrast enhancement requirement for the plurality of local regions in the key grayscale image of the TMJ surgical video and the corresponding time node for the grayscale image of the TMJ surgical video is specifically implemented based on the following equation:
$$J {L}_{v} = J {B}_{v} \times \{ 1 + t h [ ( {t}_{v} - {t}_{s} ) \times ( {t}_{a} - {t}_{v} ) ] \}$$
wherein, JLᵥ represents a resolution enhancement credibility of the v^{th} key grayscale image of the TMJ surgical video; JBᵥ represents the contrast enhancement requirement for the local regions in the v^{th} key grayscale image of the TMJ surgical video; tᵥ represents a time node for the v^{th} key grayscale image of the TMJ surgical video; tₛ represents a time node for a first grayscale image of the TMJ surgical video; tₐ represents a time node for a last grayscale image of the TMJ surgical video; and th[ ] is a hyperbolic tangent function.

2. The construction method of an AI-simulated teaching model for TMJ surgery according to claim 1, wherein the obtaining a plurality of key grayscale images of the TMJ surgical video according to differences between grayscale values of pixels in adjacent grayscale images of the TMJ surgical video specifically comprises:
obtaining an information difference between the adjacent grayscale images of the TMJ surgical video according to the differences between the grayscale values of the pixels in the adjacent grayscale images of the TMJ surgical video and a difference between information entropies for the grayscale values of the pixels; and
obtaining the plurality of key grayscale images of the TMJ surgical video according to the information difference between the adjacent grayscale images of the TMJ surgical video.

3. The construction method of an AI-simulated teaching model for TMJ surgery according to claim 2, wherein the obtaining an information difference between the adjacent grayscale images of the TMJ surgical video according to the differences between the grayscale values of the pixels in the adjacent grayscale images of the TMJ surgical video and a difference between information entropies for the grayscale values of the pixels is specifically implemented based on the following equation:
$${JV}_{i , i + 1} = \frac{1}{n} {\sum }_{j = 1}^{n} \vert {I}_{j}^{i} - {I}_{j}^{i + 1} \vert \times \vert {H}_{i} - {H}_{i + 1} \vert$$
wherein, JV_{i, i+1} represents an information difference between an i^{th} grayscale image and an (i+1)^{th} grayscale image of the TMJ surgical video; n represents a quantity of all pixels in the grayscale images of the TMJ surgical video;
$${I}_{j}^{i}$$
represents a grayscale value of a j^{th} pixel in the i^{th} grayscale image of the TMJ surgical video;
$${I}_{j}^{i + 1}$$
represents a grayscale value of a j^{th} pixel in the (i+1)^{th} grayscale image of the TMJ surgical video; Hᵢ represents an information entropy for gray values of all pixels in the i^{th} grayscale image of the TMJ surgical video; Hᵢ₊₁ represents an information entropy for gray values of all pixels in the (i+1)^{th} grayscale image of the TMJ surgical video; and ∥ is an absolute value function.

4. The construction method of an AI-simulated teaching model for TMJ surgery according to claim 2, wherein the obtaining the plurality of key grayscale images of the TMJ surgical video according to the information difference between the adjacent grayscale images of the TMJ surgical video specifically comprises:
in all grayscale images of the TMJ surgical video, sorting all information differences between two adjacent grayscale images of the TMJ surgical video in an ascending manner to obtain an information difference sequence; and
in the information difference sequence, calculating an absolute value of a difference between two adjacent information differences, and labeling two grayscale images of the TMJ surgical video corresponding to a former information difference in two information differences that correspond to a maximum value in all absolute values of differences between adjacent information differences as key grayscale images of the TMJ surgical video.

5. The construction method of an AI-simulated teaching model for TMJ surgery according to claim 1, wherein the obtaining visual quality of each labeled patch in the key grayscale image of the TMJ surgical video according to differences between grayscale values of pixels in the labeled patches and the unlabeled patches in the key grayscale image of the TMJ surgical video is specifically implemented based on the following equation:
$$J {H}_{e}^{v} = {\sum }_{f = 1}^{{U}^{v}} \vert {\overline{{h}_{e}}}^{v} - {\overline{{h}_{f}}}^{v} \vert$$
wherein, JHₑ^{v} represents the visual quality of the e^{th} labeled patch in the v^{th} key grayscale image of the TMJ surgical video; U^{v} represents a quantity of all unlabeled patches in the v^{th} key grayscale image of the TMJ surgical video; h̅ₑ̅^{v} represents an average grayscale value for all pixels in the e^{th} labeled patch in the v^{th} key grayscale image of the TMJ surgical video; h̅_{f}^{v} represents an average grayscale value for all pixels in an f^{th} unlabeled patch in the v^{th} key grayscale image of the TMJ surgical video; and ∥ is an absolute value function.
